(19) United States
(12) Patent Application Publication
(10) Pub. No.: US 2021/0016204 A1
(43) Pub. Date: Jan. 21, 2021

(54) PROCESS FOR REMOVING METHOXYETHANOL FROM A MIXTURE COMPRISING METHOXYETHANOL AND MORPHOLINE

(71) Applicant: BASF SE, Ludwigshafen am Rhein (DE)

(72) Inventors: Thomas HEIDEMANN, Ludwigshafen am Rhein (DE); Eva KOCH, Ludwigshafen am Rhein (DE); Barbara BECKER, Ludwigshafen am Rhein (DE); Lydia NIKOLIC, Ludwigshafen am Rhein (DE); Inna SCHWABAUER, Ludwigshafen am Rhein (DE); Jutta BICKELHAUPT, Ludwigshafen am Rhein (DE); Antje GUETTLER, Ludwigshafen am Rhein (DE); Claudia OEZKOZANOGLU, Ludwigshafen am Rhein (DE); Uwe HARNISCH, Ludwigshafen am Rhein (DE)

(21) Appl. No.: 16/980,879

(22) PCT Filed: Mar. 7, 2019
(86) PCT No.: PCT/EP2019/055676

§ 371 Date: Sep. 15, 2020

(30) Foreign Application Priority Data
Mar. 15, 2018 (EP) 18161945.3

(51) Int. Cl. B01D 15/26; C07D 295/023; B01D 15/20; B01J 20/04; B01J 20/18; B01J 20/34; C01F 7/16

(52) U.S. Cl. CPC B01D 15/265; C07D 295/023; B01D 15/203; B01J 20/041; C01P 2002/72; B01J 20/3466; C01F 7/162; C01P 2002/32; B01J 20/18

(57) ABSTRACT

A method for removing methoxyethanol from a mixture comprising methoxyethanol and morpholine makes use of the selective adsorption of methoxyethanol onto a mixed oxide comprising a spinel phase. The mixed oxide comprises 20 to 30% by weight MgO and 80 to 70% by weight Al₂O₃. The spinel phase has the formula MgAl₂O₄. The mixture is a pre-purified reaction output of the reaction of diethylene glycol with ammonia in the presence of an amination catalyst.

The present invention relates to a method for removing methoxyethanol from a mixture comprising methoxyethanol and morpholine by selective adsorption.

WO 2009/080507 A1 describes a method for preparing an amine by reacting a primary or secondary aldehyde and/or ketone with hydrogen and a nitrogen compound selected from ammonia, primary amines and secondary amines in the presence of a catalyst comprising zirconium dioxide, copper and nickel. A method for preparing morpholine is described as an example. In this case, diethylene glycol is dehydrogenated and the resulting aldehyde is reacted with ammonia with elimination of water and subsequent hydrogenation to give an amine which is cyclized to give morpholine. However, in an undesired parallel reaction, the aldehyde is decarbonylated and methoxyethanol is formed. The mixture of reaction products must therefore be subsequently purified, for example by fractional rectification under reduced pressure, standard pressure or elevated pressure.

Suitable workup processes are described, for example, in EP-A-1 312 600 and EP-A-1 312 599. Low-boiling and high-boiling fractions are separated successively by distillation and the resulting amine-containing mixture is extracted with sodium hydroxide solution. An aqueous phase comprising sodium hydroxide solution and a second aqueous organic phase comprising the amine are obtained. Subsequent distillation of the aqueous organic phase results in anhydrous amine or an amine/water azeotrope which must be further purified.

WO 2008/037589 A1 describes a method for the continuous distillative separation of mixtures comprising morpholine, monoaminodiglycol, ammonia and water.

CN 104262177 A describes a method for separating a crude product consisting of diglycolamine, morpholine, diglycol and impurity by column chromatography.

The known methods for purifying the mixtures comprising methoxyethanol and morpholine are very complex and are associated with a considerable outlay in terms of apparatus and a significant energy expenditure. One problem is the very similar physical properties of methoxyethanol and morpholine. This makes the complete separation of methoxyethanol and morpholine difficult which in turn leads to a residual content of methoxyethanol remaining in the morpholine. This can lead to problems with respect to specification and product quality.

The object of the present invention is therefore to specify a method for removing methoxyethanol from a mixture comprising methoxyethanol and morpholine and to remedy one or more disadvantages of the prior art.

It has now been found that methoxyethanol is more strongly adsorbed onto mixed oxides comprising a spinel phase than morpholine. Presumably methoxyethanol is able to be adsorbed onto crystallite surfaces of the spinel phase via a bidentate binding whereas morpholine, by virtue of its structure and conformation, is not capable of this.

The invention therefore relates to a method for removing methoxyethanol from a mixture comprising methoxyethanol and morpholine by selective adsorption of methoxyethanol onto a mixed oxide comprising a spinel phase.

The mixture comprising methoxyethanol and morpholine is generally a reaction output of the reaction of diethylene glycol with ammonia in the presence of an amination catalyst. The method according to the invention is particularly suitable for fine purification of morpholine which has been pre-purified by other methods. Typically, the primary reaction output is purified by distillation. In a first step ammonia is removed, in a second step water of reaction and low-boiling by products are removed, in a third step the morpholine is separated off and the fourth step is a morpholine fine distillation. The mixture obtained in the fine distillation is a suitable starting material for the method according to the invention.

The pre-purified reaction output thus obtained generally comprises, in addition to methoxyethanol and morpholine, at least one component selected from 1,2-ethylenediamine, methoxyethylmorpholine and formylmorpholine. The mixture comprising methoxyethanol and morpholine, which serves as starting material for the method according to the invention, comprises preferably less than 0.5% by weight, especially less than 0.3% by weight, of components other than methoxyethanol and morpholine. It comprises preferably less than 0.5% by weight, especially less than 0.3% by weight methoxyethanol, based on the total weight of methoxyethanol and morpholine. In general, it comprises 0.05% by weight or more methoxyethanol.

The mixed oxide used in accordance with the invention comprises a spinel phase. The spinel phase is preferably supported by a foreign oxide or carrier oxide. The foreign oxide has a stoichiometry different from the spinel stoichiometry and/or a structure different from the spinel structure. The foreign oxide is X-ray amorphous for example. Small crystallites of the spinel phase are generally dispersed in the mixed oxide. The average crystallite size of the spinel phase is preferably 5 nm or less. The determination of the average crystallite size is accomplished, for example, by evaluating the half-height width of the characteristic reflections in the X-ray powder diffractogram according to the Scherrer equation or according to the Rietveld method.

In the context of the present invention, spinels are understood to mean mixed metal oxides in which the oxide ions adopt cubic close sphere packing and the unit cell comprises 32 oxygen ions. Of the 64 tetrahedral sites in the unit cell, in the ideal case 8 tetrahedral sites are occupied by divalent cations A, and of the 32 octahedral sites in the unit cell, in the ideal case 16 octahedral sites are occupied by trivalent cations B, resulting in the idealized formula AB₂O₄ (Ullmann's Encyclopedia of Industrial Chemistry, 6th edition, 2003, Wiley-VCH Verlag GmbH & Co. KGaA, Weinheim, Volume 26, pages 651-652).

In the formula

AB₂O₄

A is a divalent cation, preferably Mg, Fe, Co, Ni, Mn, Zn or Cd; and

B is a trivalent or tetravalent cation, preferably Al, Fe, Co, Cr, Ga, La or Ti.

In the context of the present invention, spinels also include those metal oxides which deviate from the ideal formula and can be described by the phases A₁₋ₓB₂₋ₓO₄, where x can assume the values of 0<x≤1 and at the same time the molar ratio of total metal to oxygen is 3 to 4.

The spinel phase particularly preferably has the idealized formula MgAl₂O₄.

The mixed oxide preferably essentially exclusively comprises oxides of magnesium and aluminum. The mixed oxide preferably comprises 20 to 30% by weight MgO and 80 to 70% by weight Al₂O₃, especially 25 to 27.5% by weight MgO and 75 to 82.5% by weight Al₂O₃.

The X-ray powder diffractogram of the mixed oxide is preferably characterized by reflections at the interplanar spacings d [Å]=4.61±0.1; 2.86±0.1; 2.43±0.1; 2.01±0.1; 1.56±0.1 and 1.42±0.1. Owing to the small crystallite size, the reflections may be broad and poorly resolved.

The X-ray reflections in this application are reported in the form of the interplanar spacings d [Å] independent of the wavelength of the X-ray radiation used. The wavelength λ of the X-ray radiation used for the diffraction and the diffraction angle θ (the reflection position used is the peak of a reflection in the 2θ plot) are related to each other by the Bragg relationship as follows:

2 sin θ=λ/d

where d is the interplanar spacing which corresponds to the particular reflection in the three-dimensional atom arrangement.

The mixed oxide is particulate, for example in the form of spheres, rings, tablets, extrudates or chippings. For example, the particles have an average particle size (in the direction of the largest spatial expansion) of 1 to 30 mm, preferably 5 to 20 mm. For example, it is possible to use an extrudate having a diameter of 1.3 to 1.5 mm and a length of 5 to 20 mm.

The mixed oxides are prepared according to methods known per se, for example by co-precipitation of the mixed aqueous solutions of metal sources, such as metal nitrates, with an aqueous solution of an alkali metal hydroxide and/or alkali metal carbonate and subsequent calcination (W. Xu, Xi Liu, J. Ren, H. Liu, Y. Ma, Y. Wang, G. Lu, Microporous Mesoporous Mater. 2011, 142, pages 251-257). The calcination step is generally conducted at temperatures in the range of 400 to 1000° C., preferably 600 to 900° C. The desired metal ratio is adjusted by appropriate mixing of the metal nitrate solutions.

The precipitates obtained after precipitation are further processed to afford the spinels. In addition, the precipitates are washed, wherein the content of alkali metal, supplied by the mineral base used as precipitating agent, is influenced by the amount and temperature of the wash water. Generally, the content of alkali metal is reduced when the washing time is extended or when the temperature is increased. After washing, the precipitate is dried and milled.

The washed and dried precipitate can be made into a paste with water and can be extruded. The extrudate is dried and then calcined at temperatures of 300° C. to 800° C., preferably at about 600° C.

Alternatively, the mixed oxide can be compressed to give shaped bodies. The shaping is preferably effected by tableting. Tableting is a method of press agglomeration. In this case, a pulverulent bulk material or bulk material previously agglomerated in a press mold is charged between two punches with a so-called matrix and is compacted by single-axis compression and shaped to give a solid tablet. The tableting is effected, for example, by so-called rotary presses or eccentric presses. During the tableting, tableting aids such as graphite or magnesium stearate may be used.

For the selective adsorption, the mixture comprising methoxyethanol and morpholine is brought into contact with the mixed oxide, preferably by passing the mixture over a bed of the mixed oxide. For this purpose, the mixed oxide is suitably in the form of a fixed bed arranged in an adsorption column through which the substance stream is passed. The adsorption column is preferably arranged vertically through which the substance stream flows in the gravitational direction or counter to the direction of gravitational force. The dimension of the fixed bed in the flow direction is preferably 2 to 15 fold that of the (longest) diameter of the fixed bed. Several adsorption columns may also be used connected in series. The morpholine discharging from the adsorption column has a reduced content of methoxyethanol compared to the mixture introduced to the column.

It has been found that the adsorptive separation of methoxyethanol and morpholine on the mixed oxide proceeds with better separation sharpness in the absence of water. It is therefore preferable to dry the mixture prior to the selective adsorption. In a preferred embodiment, the mixture is dried by being brought into contact with a molecular sieve. Suitable molecular sieves have an average pore size of about 0.3 to 0.4 nm. For instance, the mixture can be passed over a fixed bed which, upstream to the flow direction of the mixture, comprises a molecular sieve in a first zone and comprises a mixed oxide defined above in a second zone downstream. The substance stream firstly comes into contact with the molecular sieve in a first zone, wherein water is preferably adsorbed. Relatively large oxygen-containing or nitrogen-containing molecules are adsorbed with lower preference in this first zone. Only in the subsequent second zone is methoxyethanol adsorbed with preference over morpholine onto the mixed oxide. The embodiment described, in which preferably water is removed in a first zone, has the advantage that—even when saturation of the mixed oxide is quite advanced—no displacement of methoxyethanol already adsorbed by water takes place.

The mixed oxide is preferably dried prior to bringing into contact with the mixture comprising methoxyethanol and morpholine. The drying can be carried out by passing over a dry inert gas, preferably nitrogen gas, at elevated temperature. Suitable temperatures are 90 to 200° C., especially 100 to 150° C. The drying of the mixed oxide can be carried out in several stages at increasing temperature. Subsequently, dry inert gas is passed over the mixed oxide until this has cooled down. If a structured bed of molecular sieve and mixed oxide is used, as described above, preferably both zones are dried by passing over the dry inert gas at elevated temperature.

After an operating period the mixed oxide is saturated, i.e. its surface is occupied with methoxyethanol, and the adsorption becomes increasingly non-selective or no further adsorptive removal of methoxyethanol from the substance stream takes place during passage of the substance stream. The mixed oxide is then preferably regenerated. Advantageously, at least two adsorption columns may be provided, of which a first column is in the adsorption cycle while the other column is being regenerated. If the mixed oxide of the first column is saturated, the substance stream is diverted and passed through the second adsorption column so that the mixed oxide in the first column can be regenerated.

The mixed oxide is suitably regenerated by treatment with water. In this case, adsorbed methoxyethanol is washed off the mixed oxide. Subsequently, the mixed oxide is dried, as described above. The washing of the mixed oxide is preferably effected using 5 to 10 wash fractions, wherein one wash fraction corresponds to the volume of the adsorbent bed. The washing is carried out at room temperature, but in wash fractions 3, 4 and 5 the reactor is preferably heated to 80° C. and in each case allowed to stand for one hour filled with water.

In order to minimize morpholine losses and the loading of the wash water by morpholine, which has been coadsorbed onto the mixed oxide, it is preferable to desorb coadsorbed morpholine prior to the regeneration, preferably by passing over an inert gas, such as nitrogen gas, or an inert gas containing steam, such as in particular moist nitrogen gas. The desorption of the coadsorbed morpholine is carried out preferably by heating the mixed oxide to an elevated temperature of, for example, 50 to 150° C., especially 50 to 100° C. Temperatures up to 100° C. are preferred since higher temperatures can lead to discoloration of the recovered morpholine. The morpholine can be condensed out from the charged inert gas stream.

The invention is more particularly elucidated by the appended figure and the examples which follow.

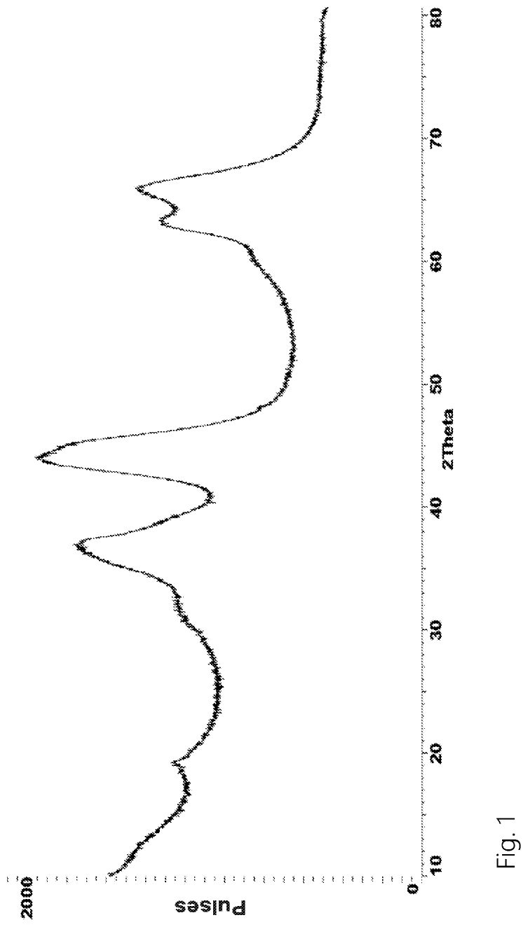

FIG. 1 shows the X-ray powder diffractogram of the mixed oxide according to example 2.

### EXAMPLES

The XRD analyses were carried out using a D8 Advance Series 2 from Bruker/AXS using a CuK-alpha source (having a wavelength of 0.154 nm at 40 kV and 40 mA). The measurements were carried out over the measuring range: 10-80° (2Theta), 0.02° steps at 2.4 seconds/step. To determine the average crystallite sizes of the individual phase, the TOPAS (Bruker AXS) structure analysis software was used.

### Example 1: Preparation of a Spinel-Containing Mixed Oxide Consisting of 25% MgO and 75% Al₂O₃

An aqueous solution (1.95 L), comprising 628.23 g of magnesium nitrate and 1889.2 g of aluminum nitrate, was simultaneously precipitated with a 20 percent aqueous sodium carbonate solution at 80° C. in a stirred vessel in a constant stream such that the measured pH was maintained at 5.5. The pH was then adjusted to pH 7.8 with a sodium carbonate solution and the reaction solution was further stirred for circa 30 minutes. The resulting suspension was filtered and washed with water until the conductivity of the filtrate was about 50 μS and then dried at 100° C. for 16 hours. The powder was milled to a particle size of below 500 μm. An extrudate in the form of 1.5 mm length strands was produced from the powder at a pressure of 80 bar with 70 mL of water and at a kneading time of 70 min. The resulting extrudate was dried at 120° C. for 16 hours and subsequently calcined at 600° C. for 1 hour at a heating rate of 2° C./min.

### Example 2

The spinel was produced as in example 1 but a different ratio of magnesium nitrate and aluminum nitrate solutions was used. Thus, a spinel was obtained having the composition of 27.5% MgO and 72.5% Al₂O₃.

### Example 3

Adsorption of Methoxyethanol (MeOEtOH) Onto the Spinel from Example 1

A laboratory column was packed with a molecular sieve (100 mL) and the spinel (80 mL), in which the molecular sieve was packed prior to the spinel. Subsequently, the molecular sieve and the adsorbent were dried in two steps, where nitrogen was initially passed through (20 NL/h) at 100° C. for 20 hours and then at 150° C. for 6 hours. Morpholine comprising about 0.1% by weight methoxyethanol was passed through the dry adsorbent material. The adsorption was carried out at room temperature at a flow rate of 10 g/h to 15 g/h. Samples were regularly analyzed by means of gas chromatography and a reduction of the methoxyethanol content by 50% was defined as breakthrough.

The results are shown in the following table.

| | | Cumulative | |
|---|---|---|---|
| | | adsorbent loading | Decrease of |
| | Methoxyethanol | kg_{MeOEtOH}/ | methoxyethanol |
| Sample | area % | t_{adsorbent} | [%] |
| 1 | 0.0310 | 3.48 | 71.52 |
| 2 | 0.0194 | 8.05 | 82.17 |
| 3 | 0.0240 | 11.10 | 78.02 |
| 4 | 0.0433 | 16.43 | 60.23 |
| 5 | 0.0601 | 20.01 | 44.86 |
| 6 | 0.0764 | 24.91 | 29.93 |

The capacity of the adsorbent was about 17 kg_{MeOEtOh}/t_{adsorbent}.

### Example 4

Adsorption of Methoxyethanol Onto the Spinel from Example 2

The adsorption experiments were performed analogously to example 3. The results are shown in the following table.

| | | Cumulative | |
|---|---|---|---|
| | | adsorbent loading | Decrease of |
| | Methoxyethanol | kg_{MeOEtOH}/ | methoxyethanol |
| Sample | area % | t_{adsorbent} | [%] |
| 1 | 0.008 | 3.60 | 92.66 |
| 2 | 0.000 | 5.85 | 100.00 |
| 3 | 0.006 | 9.50 | 94.50 |
| 4 | 0.061 | 14.15 | 44.04 |
| 5 | 0.087 | 17.30 | 20.18 |

The capacity of the adsorbent was about 14 kg_{MeOEtOh}/t_{adsorbent}.

### Example 5

Regeneration of the Spinel from Example 1

The spinel from example 1 was regenerated by washing with water at room temperature. For this purpose, 10 wash fractions were used, in which one wash fraction corresponds to one bed volume and the flow rate was 400 g/h. Each wash fraction was analyzed by gas chromatography and the mixed oxide was subsequently dried with nitrogen (20 NL/h; 2 d at 80° C., 2 h at 100° C., 2 h at 120° C., 6 h at 150° C.).

The results of the gas chromatography (area%) are shown in the following table. In this case, it was clear that the wash water still comprised considerable amounts of morpholine.

| | Morpholine content |
|---|---|
| Fraction | area % |
| 1 | 30.48 |
| 2 | 7.46 |
| 3 | 0.49 |
| 4 | 0.07 |
| 5 | 0.02 |
| 6 | 0.02 |
| 7 | 0.02 |
| 8 | 0.01 |
| 9 | 0.01 |
| 10 | 0.01 |

### Example 6

Regeneration of the Spinel from Example 2

The spinel was regenerated by a different process.

In the first regeneration, the column was heated to 50° C. to 80° C. and dry nitrogen was passed through. Subsequently, the charged nitrogen was passed over a cool condensor at 5° C., whereupon morpholine separated out. The adsorbent was then washed with 5 wash fractions of water and dried with nitrogen. The morpholine which separated out and the wash fractions were analyzed by means of gas chromatography.

The second regeneration was carried out in analogy to the first, with the difference that the column was heated to 50° C. to 150° C. The morpholine separated out on the condensor and the wash fractions were analyzed by means of gas chromatography. Morpholine loss was estimated from the data.

In the case of the third regeneration, the column was heated to 50° C. to 100° C. and, after passing through the dry nitrogen, was treated with nitrogen that had been moistened by means of a water bottle which comprised water at room temperature. The subsequent regeneration was carried out in analogy to the second regeneration, in which the morpholine separated off and the wash fractions were analyzed by means of gas chromatography.

The fourth regeneration was carried out in analogy to the third, with the difference that the nitrogen had been moistened with warm water at 90° C.

| | Morpholine loss |
|---|---|
| Regeneration | % |
| 1 | 0.34 |
| 2 | 0.18 |
| 3 | 0.21 |
| 4 | 0.11 |

1. A method for removing methoxyethanol from a mixture comprising methoxyethanol and morpholine by selective adsorption of methoxyethanol onto a mixed oxide comprising a spinel phase.

2. The method according to claim 1, wherein the spinel phase has the formula
AB₂O₄
in which
A is a divalent cation; and
B is a trivalent or tetravalent cation.

3. The method according to claim 2, wherein the spinel phase has the formula MgAl₂O₄.

4. The method according to claim 3, wherein the mixed oxide comprises 20 to 30% by weight MgO and 80 to 70% by weight Al₂O₃.

5. The method according to claim 1, wherein the mixture is passed over a bed of the mixed oxide.

6. The method according to claim 1, wherein the mixture comprises in addition at least one component selected from 1,2-ethylenediamine, methoxyethylmorpholine and formylmorpholine.

7. The method according to claim 1, wherein the mixture is dried prior to the selective adsorption.

8. The method according to claim 7, wherein the mixture is dried by bringing it into contact with a molecular sieve.

9. The method according to claim 1, wherein the mixed oxide is regenerated by treatment with water.

10. The method according to claim 9, wherein coadsorbed morpholine is desorbed prior to the regeneration of the mixed oxide.

11. The method according to claim 10, wherein coadsorbed morpholine is desorbed by passing over an inert gas or an inert gas containing steam.

12. The method according to claim 11, wherein the desorbed coadsorbed morpholine is condensed out from the inert gas or inert gas containing steam.

13. The method according to claim 2, wherein
A is Mg, Fe, Co, Ni, Mn, Zn or Cd; and
B is Al, Fe, Co, Cr, Ga, La or Ti.

14. The method according to claim 3, wherein the mixed oxide comprises 25 to 27.5% by weight MgO and 75 to 82.5% by weight Al₂O₃.
